(19) United States
(12) Patent Application Publication
(10) Pub. No.: US 2024/0261527 A1
(43) Pub. Date: Aug. 8, 2024

(54) NASAL CANNULA

(71) Applicant: EFFORTLESS OXYGEN, LLC, Phoenix, AZ (US)

(72) Inventors: Joel B. METELITS, Scottsdale, AZ (US); Lisa M. UPHAM, LeRaysville, PA (US); Samir S. AHMAD, San Diego, CA (US)

(21) Appl. No.: 18/435,893

(22) Filed: Feb. 7, 2024

Related U.S. Application Data
(60) Provisional application No. 63/443,914, filed on Feb. 7, 2023.

(51) Int. Cl. A61M 16/06

(52) U.S. Cl. CPC A61M 16/0672; A61M 16/0683

(57) ABSTRACT

A nasal cannula system including a nasal cannula for delivery of oxygen to the nostrils of a patient, and a headgear. The oxygen enters the interface on the side of the head and above or below the ear. The system uses flat tubing made of soft material that is comfortable on the face, exerting less pressure on the skin. The system reduces the noise of the oxygen by entering a cannula having an expansion volume before it enters the nostrils. The cannula is secured above and away from the ears by a dual strapped headgear. The dual straps provide greater stability for the interface. The straps attach through a slit on the back of the triangular pad. The straps are able to be adjusted on each side of the head for comfort and stability.

### CROSS-REFERENCE TO RELATED APPLICATION

This application claims priority to U.S. Provisional Application Ser. No. 63/443,914, filed Feb. 7, 2023, the contents of which are incorporated herein in their entirety, by reference.

### BACKGROUND OF THE DISCLOSURE

This disclosure relates generally to nasal cannula systems, and more particularly, to a nasal cannula.

### BACKGROUND AND SUMMARY

Conventional oxygen nasal cannulas generally consist of tubes and other means for delivering therapeutic gases adapted for use with the nasal passage of a patient.

In such conventional oxygen nasal cannulas, the gas is transported by a thin flexible main tube made of an inert material. The main tube terminates at an opening which can be inserted into the patient's nostrils. Typically, a pair of smaller nasal insert tubes protrude from the main tube, or the main tube splits at a Y-junction into two smaller tubes. Each smaller nasal insert tube is generally inserted some distance into a nasal cavity to carry gas to the nostril, thereby increasing the fraction of inspired oxygen.

Conventional nasal oxygen cannulas usually loop around the ears to hold the cannula in place. But in order to minimize leakage of the delivered gas the loops around the ears must be sufficiently tight. Also, the cannula tubing exerts uncomfortable pressure on the patient's face, resulting in such cannula tubing to be rather constraining and uncomfortable for the patient.

Additionally, many oxygen cannula users are susceptible to skin breakdown of the ears. Positioning of the gas delivery tubes over the ears of the wearer also may lead to skin breakdown and pressure ulcers. See, Turyanica et al, Inadence Correlates and Interventions Used for Pressure Ulcers of the Ears, MEDSURG Nursing, September-October 2011, Vol. 20/No. 5, pages 241-246, and Black et al., Medical Device-Related Pressure Ulcers, Chronic Wound Care Management and Research 2016; 3, pages 91-93.

Also, nasal cannulas are somewhat noisy, generating sounds from turbulent flow, and sounds created every time a bolus of gas is released. Sound generated can be annoying to both the wearer and caretakers and other persons within hearing distance of the wearer.

The present disclosure overcomes the aforesaid and other disadvantages of the prior art by providing a unique headgear system configured to support the nasal cannula, securing the nasal cannula to the patient. The unique headgear of the present disclosure avoids running cannula tubing from behind or over the ears and includes a noise-reducing hollow body located in line between the fluid source and the nasal cannula. The nasal cannula system also includes at least one nostril tube insert configured to extend inside of the patient's nasal cavity.

In one embodiment of the disclosure, we provide a nasal cannula assembly adapted to deliver a gas flow from a supply tube to a patient, said nasal cannula having at least one nasal tube configured to be inserted into a patient's nostril, and an enlarged hollow body relative to the supply tube, connecting the supply tube to the at least one nasal tube.

Preferably the supply tube is configured to be connected to a gas supply. The gas supply may comprise a portable gas tank, a stationary gas tank, an oxygen concentrator, and a plumbed central system. In one embodiment the gas comprises oxygen enriched air.

Preferably the supply tube and cannula are supported by a headgear system configured to rest on the back and a top of a head of the patient.

In one embodiment the headgear system comprises two or more head straps.

In one embodiment the head straps are connected together at a triangular pad which also supports the supply tube.

In another embodiment the head straps and/or triangular pad include a breathable cloth liner on an underside thereof and may include an adjustable clip.

In another embodiment the supply tube has a flat side configured to face skin of the patient.

In a further embodiment the head straps comprise two head straps joined at a single strap extension. In such embodiment, the single strap extension may include a slide clasp for adjusting length of the single strap extension.

In yet another embodiment the supply tube is supported by the single strap extension.

In a further embodiment the headgear system is configured to hold the supply tube away from contact with an ear of the patient.

In yet another embodiment, there is provided a cannula assembly adapted to deliver a gas flow from a supply tube to a patient, and nasal cannula having at least one nasal tube configured to be inserted into a patient's nostril, and a headgear system configured to rest on a back and a top and/or side of a head of a patient, wherein the headgear system is configured to hold the supply tube away from contact with an ear of the patient.

In another embodiment the supply tube is configured to run up the back of a head of the patient.

The present disclosure also provides a method for reducing sound of gas flow being supplied via a nasal cannula, comprising providing between a source of the gas flow and the nasal cannula, an enlarged hollow body.

In a preferred embodiment the enlarged hollow body is provided by a supply tube between the source of gas and the nasal cannula.

### BRIEF DESCRIPTION OF THE DRAWINGS

Further features and advantages of the present disclosure will be seen from the following detailed description, wherein:

FIG. 1 is a perspective view of a first embodiment of a nasal cannula system in accordance with the present disclosure; and

FIG. 2 is a view similar to FIG. 1 of an alternative embodiment of nasal cannula system in accordance with the present disclosure and showing the head strap in partial cross-section.

### DETAILED DESCRIPTION OF THE DISCLOSURE

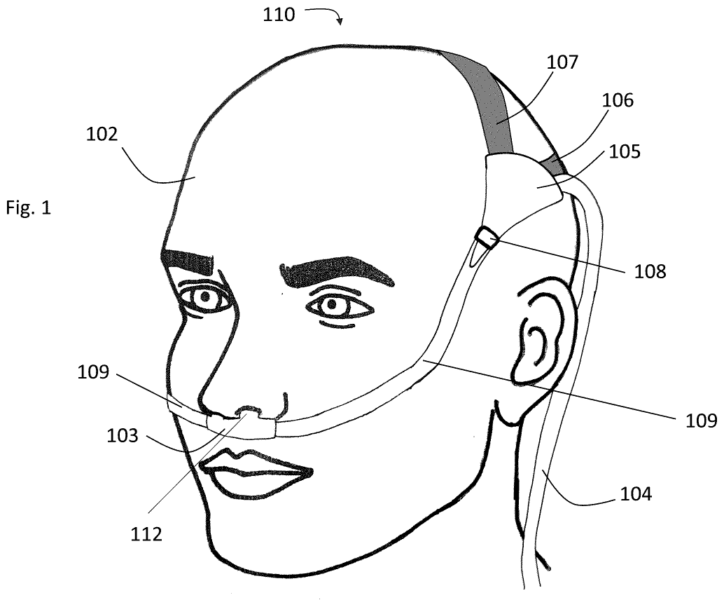

FIG. 1 illustrates patient 102 wearing a first embodiment of a nasal cannula system according to the present disclosure. The nasal cannula system includes a nasal cannula 103 and a headgear 110 that includes supply tubes 109 and a head gear system that includes head straps and a connection pad as will be described below. The nasal cannula 103 has at least one nostril tube 112 configured for insertion into a patient's nostrils. Nasal cannula 103 comprises an enlarged hollow body relative to the supply tubes 109. That is to say the nasal cannula 103 hollow body should be larger in interior cross-section than the supply tubes in order to provide an expansion space for the gas or oxygen as it is being delivered to the nostril tube(s), thus reducing sounds from turbulent flow and sounds created by a bolus of gas or oxygen as it is being delivered.

The nasal cannula 103 is attached to a supply tube 104 that leads to a main gas supply (not shown). The gas supply may be a portable tank, a stationary gas tank, an oxygen concentrator, or a plumbed central system.

The head gear system includes head straps 106 and 107 which are attached to a triangular pad 105 (only one of which is shown), that are configured to rest on the side or top wearer's head above the wearer's ears. The straps may include a breathable cloth linen (not shown) configured to rest on the back and the top of the head of the wearer. Head straps 106 and 107 are connected to the back of the triangular pads 105 separately or together. They may be woven through a single or multiple slots (not shown) in the triangular pad.

The triangular pads 105 may be molded and optionally pliable in nature, for example, made from a plastic material such as polypropylene. The triangular pads 105 may be covered by a breathable cloth on its surface.

The head straps 106 and 107 are configured to sit behind the triangular pads 105 or connect to the triangular pads 105 and be adjusted for comfort by weaving through slide clasps 108. The triangular pads could be replaced by adjustable clips which will allow for oxygen introduction to the system from above and behind one ear of the wearer to above and behind the opposite ear based on the patient's anatomy and comfort. The head straps 106 and 107 preferably are made from a flexible material such as neoprene.

Supply tubes 109 are flat or round tubes and are connected to the oxygen source tube 104 through the triangular pads 105 or attached by a clip. As a practical matter only one oxygen source tube 104 is required. Thus, the supply tube 109 on the other side of the head gear may dead end at the triangular pad 105. Alternatively, supply tube 109 on the other side of the head may comprise a solid tube or a strap.

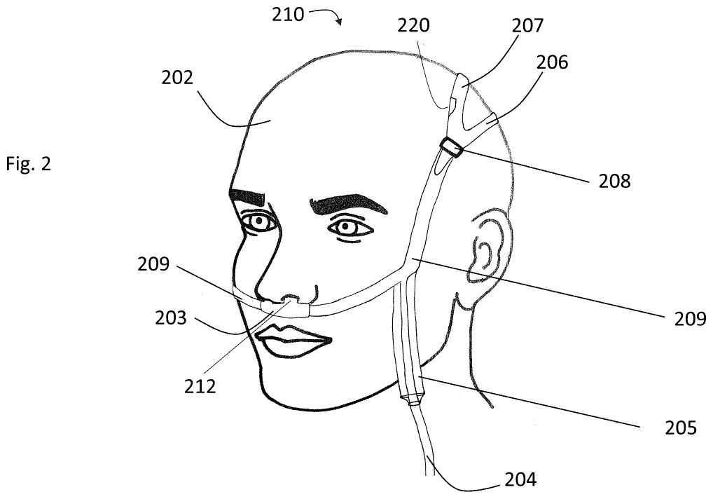

FIG. 2 illustrates an alternative embodiment of the disclosure and shows a patient 202 using an embodiment of the nasal cannula with headgear 210. As before, a nasal cannula 203 having at least one nostril tube 212 inserted into the patient's nostrils is attached to a flat or round supply tube 205 which connects to an oxygen tube 204 which in turn is connected to a gas supply (not shown). Oxygen supply tube 204 is threaded through flat or round tubing 205 which also connects to hollow head gear straps 209. Head gear straps 209 are flat or round somewhat rigid material that connects directly to, or via a clip, to headgear straps 206 or 207.

The headgear includes two straps 206 and 207. As in the case of the FIG. 1 embodiment straps 206 and 207 may include a breathable cloth linen (shown at 220) resting on the back and the top of the head. Head straps 206 and 207 are joined into a single strap that can be adjusted for comfort and stability at slide clasps 208. As in the case of the FIG. 1 embodiment, only one side of the head gear is visible in the drawings figure. However, it will be understood that the head gear straps 206, 207, and 209 continue around the other side of the patient's head and meet at a second slide clasp 208. The head straps 206 and 207 are preferably made from a flexible material such as neoprene.

A feature and advantage of the present disclosure includes increased patient comfort because of the lack of tubing wrapped around the ears of the patient. Thus, the possibility of skin breakdown and pressure ulcers around the ears is eliminated. The tubing also is not tightly cinched up uncomfortably under the chin as in the case of some prior art nasal cannula systems.

Another advantage of the head gear of the present disclosure is the ability to maintain the tubing within the nostrils. The headgear straps can be adjusted to maintain stability and comfort.

Yet another advantage is to reduce the noise by increasing the internal volume size of the nasal cannula to provide an expansion volume having at least one oxygen inlet and at least one nostril tube insert.

Still yet another advantage results from the use of round tubing within a flat soft textured PVC material toward the skin made to reduce facial skin pressure and increase comfort. The oxygen entry tube will enter or clip into the triangular pad above the ear and below the lower head strap, allowing for nearly 180 degree positioning.

Additionally, the enlarged hollow body between the supply tubes and the nostril tube(s) provides an expansion space for the oxygen as it is delivered to the nostril tubes, thus reducing sounds from turbulence flow and sounds created by a bolus of oxygen as it is being delivered.

What is claimed:

1. A nasal cannula assembly adapted to deliver a gas flow from a supply tube to a patient, said nasal cannula having at least one nasal tube configured to be inserted into a patient's nostril, and
an enlarged hollow body relative to the supply tube, connecting the supply tube to the at least one nasal tube.

2. The nasal cannula assembly of claim 1, wherein the supply tube is configured to be connected to a gas supply.

3. The nasal cannula assembly of claim 2, wherein the gas supply is selected from the group consisting of a portable gas tank, a stationary gas tank, an oxygen concentrator and a plumbed central system.

4. The nasal cannula assembly of claim 1, wherein the gas comprises oxygen enriched air.

5. The nasal cannula assembly of claim 1, wherein the supply tube and cannula are supported by a headgear system configured to rest on a back and a top and/or sides of a head of the patient.

6. The nasal cannula assembly of claim 5, wherein the head gear system comprises two or more head straps.

7. The nasal cannula assembly of claim 6, wherein the head straps are connected together at a triangular pad which also supports the supply tube.

8. The nasal cannula assembly of claim 7, wherein the straps and/or triangular pad include a breathable cloth liner on an underside thereof.

9. The nasal cannula assembly of claim 7, wherein the triangular pad includes an adjustable clip.

10. The nasal cannula assembly of claim 1, wherein the supply tube has a flat side configured to face skin of the patient.

11. The nasal cannula assembly of claim 5, wherein the head straps comprise two head straps joined at a single strap extension.

12. The nasal cannula assembly of claim 10, further comprising a slide clasp for adjusting a length of the single strap extension.

13. The nasal cannula assembly of claim 10, wherein the supply tube is supported by one of the straps.

14. The nasal cannula assembly of claim 1, wherein the supply tube is configured to avoid contact with an ear of the patient.

15. The nasal cannula assembly of claim 13, wherein the supply tube is configured to run up a back of a head of the patient.

16. A nasal cannula assembly adapted to deliver a gas flow from a supply tube to a patient, said nasal cannula having at least one nasal tube configured to be inserted into a patient's nose, and
a headgear system configured to rest on a back and a top and/or side of a head of the patient, wherein the headgear system is configured to hold the supply.

17. The nasal cannula assembly of claim 16, wherein the supply tube is configured to run up a back of a head of the patient.

18. A method for reducing sound of gas flow being supplied via a nasal cannula, comprising providing between a source of the gas flow and the nasal cannula, an enlarged hollow body.

19. The method of claim 18, wherein the enlarged hollow body is provided between a supply tube between the source of gas flow and the nasal cannula.
